(19) United States
(12) Patent Application Publication
(10) Pub. No.: US 2026/0130968 A1
(43) Pub. Date: May 14, 2026

(54) PHOTOSOLIDIFICATION METHODS AND COMPOSITIONS FOR GENERATING BIOCOMPATIBLE ARCHITECTURES

(71) Applicant: 3D Systems, Inc., Rock Hill, SC (US)

(72) Inventors: Jordan MILLER, Rock Hill, SC (US); Bagrat GRIGORYAN, Rock Hill, SC (US)

(21) Appl. No.: 19/387,095

(22) Filed: Nov. 12, 2025

Related U.S. Application Data
(60) Provisional application No. 63/719,325, filed on Nov. 12, 2024.

(51) Int. Cl. A61K 38/17; A61K 41/00

(52) U.S. Cl. CPC A61K 38/1767; A61K 41/00

(57) ABSTRACT

In one aspect, compositions and methods for making networks from solidified aqueous build materials are described herein. In some embodiments, a composition described herein comprises an aqueous build material, a photoinitiator, and a photoacid. In some cases, the aqueous build material comprises aqueous silk fibroin. Further, in some embodiments, the composition or method includes the use of a biocompatible, light-absorbing additive material configured to control light penetration. The aqueous build material is solidified upon application of an illumination beam or electric signal to desired locations of a bath comprising the aqueous build material.

### CROSS REFERENCE TO RELATED APPLICATIONS

This application claims priority pursuant to 35 U.S.C. § 119 to U.S. Provisional Patent Application No. 63/719,325, filed Nov. 12, 2024, which is hereby incorporated by reference in its entirety.

### FIELD

The present disclosure relates to compositions and methods for forming biocompatible networks, including using photosolidification of aqueous build materials.

### BACKGROUND

Bioengineered tissues and organs may be produced to restore, improve, or repair portions of tissue or whole biological tissue. However, a challenge in generating these bioengineered tissues is producing complex three-dimensional (3D) topologies that allow functionality, biomechanical stability, and vascularization similar to that of native tissue. Hydrogels can be used for 3D printed tissue structures, though production of intricate 3D features remains a challenge. Further, acrylate-based biomaterials have limitations for use in living matter, such as where radical diffusion and dark reactions lead to blooming of the reaction, which is seen at a single voxel level.

Natural materials, such as collagen, gelatin, and silk biomaterials offer greater biocompatibility and reduce risks of immune responses and toxicity. Such materials are typically prepared by casting into sheets of the material, followed by solvent evaporation, dehydration of the material, and solidification as a matrix. However, these solidification processes may be less compatible with the intricate 3D architectures required in the production of effective tissues with fluid networks. Thus, there exists a need for improved 3D printing techniques that produce articles with topological complexity but high biocompatibility.

### SUMMARY

In one aspect, methods of making biocompatible 3D networks are described herein, which in some embodiments harness the biocompatibility of natural materials and topological complexity available through light-based 3D printing. In some embodiments, such a method includes providing a bath comprising an aqueous build material and a photoinitiator, and selectively transferring heat via illumination to one or more locations of the bath. Upon heat transfer, the aqueous build material is solidified and forms a network or portion of a network with desired 3D architecture.

For reference purposes herein, the term “aqueous build material” (or its plural) can be used interchangeably with the term “ink” or “photoink” (or their plurals). In some embodiments, a bath described herein comprises any composition described herein to generate 3D networks with biocompatible materials. For example, in some cases, the bath comprises an aqueous silk fibroin build material in an amount of 5-15 wt. %, based on the total weight of the bath, and photoacid at a concentration of 1 molar (1 M) or less. In this context for purposes of determining a weight percent, it is to be understood that the “bath” refers to the materials (e.g., the liquid materials) that constitute the bath, not any container or structure that holds the bath materials.

In some embodiments, a method described herein produces a network, which comprises a vascular network, ductal network, airway network, neural network, or a combination of two or more of the foregoing. Such a network includes a 3D architecture, which may be produced by directing an illumination beam or projection generated by an optical system towards the bath based on a 3D model of the network. In some cases, the method includes directing the illumination beam or projection towards select portions of the bath held on a build plane. In such instances, the build plane may hold a first portion with one or more locations towards which the illumination beam or projection is directed to solidify the aqueous build material at the one or more locations. In some cases, the method further includes an elevator system configured to elevate the build plane to different portions of the bath to be solidified, so that directing the illumination beam or projection towards those second portions of the bath produces solidification of the aqueous build material at one or more locations of the second bath portion.

In some embodiments, the bath comprises an aqueous build material, a photoinitiator, and a photoacid. In some instances, an illumination beam or projection is directed toward a bath containing the aqueous build material, photoinitiator, and photoacid, causing the photoacid to acidify the bath in the location of heat transfer from the illumination beam or projection. In some such instances, the acidified bath solidifies the aqueous build material, such as aqueous silk fibroin, at those locations of heat transfer to form a network.

In addition, in some embodiments, photoacid is present in the bath at a concentration of 1 M or less, or at a concentration of 0.1 M or less. In some embodiments, the bath is at a temperature of 4° C. or below. In some implementations, the bath further comprises a leaching agent, which in some cases comprises one or more of tetramethylethylenediamine (TEMED), polyethylene glycol (PEG), or polyethylene glycol diacrylate (PEGDA). Moreover, in some embodiments, the photoinitiator comprises one or more of a lithium acylphosphinate, Irgacure 2959, Eosin Y system, tris(triphenlphosphine)ruthenium (II), or camphorquinone. Further, in some instances, the bath further comprises a biocompatible, light-absorbing additive material configured to control light penetration.

In another aspect, methods of forming a network by applying an electric signal to a bath comprising an aqueous build material and salt are described herein. In some such methods, a bath comprising the aqueous build material and salt is provided, as is in electrical communication with an electrode system comprising a pair of electrodes configured to generate the electric signal. When such an electric signal is selectively applied along an area of the bath, the aqueous build material at that one or more locations of the bath can be solidified and form a network or portion of a network with desired 3D architecture.

Moreover, as described further hereinabove, the 3D architecture of the network may be based on a 3D model of the network and application of the electric signal as indicated by the 3D model. Further, as described hereinabove, solidification may be undertaken at portions of the bath as held on a build plane, where an elevator system is configured to raise or lower the build plane to hold one or more different portions of the bath for application of electric signal. In such instances, the applied electric signal along areas in portions of the bath held on the build plane are solidified.

In some embodiments, the bath comprises an aqueous silk fibroin build material in an amount of 5-15 wt. %, based on the total weight of the bath. Moreover, in some embodiments, the bath further comprises a leaching agent, which may comprise one or more of TEMED, PEG, or PEGDA.

In yet another aspect, there is provided a composition configured to generate 3D, biocompatible architectures. In some embodiments, the composition comprises an aqueous build material, a photoinitiator, and a photoacid. In some instances, the aqueous build material comprises an aqueous silk fibroin build material. Moreover, in some cases, the aqueous silk fibroin is present in an amount of 5-15 wt. %, based on the total weight of the composition. Additionally, in some cases, photoacid is present in the composition at a non-zero concentration of 1 M or less, or at a concentration of 0.1 M or less. In some implementations, a composition described herein further comprises a leaching agent, which in some cases comprises one or more of TEMED, PEG, or PEGDA. Moreover, in some embodiments, the photoinitiator comprises one or more of a lithium acylphosphinate, Irgacure 2959, Eosin Y system, tris(triphenlphosphine) ruthenium (II), or camphorquinone. Further, in some instances, the composition further comprises a biocompatible, light-absorbing additive material configured to control light penetration. In addition, in some instances, the composition further comprises a salt.

Further, as described herein, in some embodiments, the article formed by a method and/or composition of the present disclosure is a network, such as a vascular network, ductal network, airway network, neural network, or a combination of two or more of the foregoing. However, other articles or networks can also be formed by a method or composition described herein.

Thus, in still another aspect, photosolidified 3D articles are described herein. Such a photosolidified 3D article can be formed from any aqueous build material and using any method described herein. Such photosolidified 3D articles, in some cases, have superior properties compared to some other 3D articles.

These and other embodiments are described in greater detail in the detailed description which follows.

### DETAILED DESCRIPTION

Embodiments described herein can be understood more readily by reference to the following detailed description and examples. Elements, apparatus and methods described herein, however, are not limited to the specific embodiments presented in the detailed description and examples. It should be recognized that these embodiments are merely illustrative of the principles of the present disclosure. Numerous modifications and adaptations will be readily apparent to those of skill in the art without departing from the spirit and scope of the disclosure.

In addition, all ranges disclosed herein are to be understood to encompass any and all subranges subsumed therein. For example, a stated range of “1.0 to 10.0” should be considered to include any and all subranges beginning with a minimum value of 1.0 or more and ending with a maximum value of 10.0 or less, e.g., 1.0 to 5.3, 1 to 4, 3 to 7, 4.7 to 10.0, 3.6 to 7.9, or 5 to 8.

All ranges disclosed herein are also to be considered to include the end points of the range, unless expressly stated otherwise. For example, a range of “between 5 and 10,” “from 5 to 10,” or “5-10” should generally be considered to include the end points 5 and 10.

Further, when the phrase “up to” (or a similar phrase) is used in connection with an amount or quantity, it is to be understood that the amount is at least a detectable amount or quantity (that is, the amount is a non-zero amount). For example, a material present in an amount “up to” a specified amount can be present from a detectable (or non-zero) amount and up to and including the specified amount.

It is also to be understood that the article “a” or “an” refers to “at least one,” unless the context of a particular use requires otherwise.

As used herein, the term “fluid network” refers to a collection of connected passageways for fluid typically found within organs and tissues, where such passageways are organized so that fluid within the fluid network is independent and not connected to (i.e., “fluidically independent” from) fluid of another fluid network. Example fluid networks may include but are not limited to the vasculature, the lymphatic system, the renal tubule system, the pancreatic ductal system, and the endoneurial/cerebrospinal fluid systems of the nervous system.

A “vascular network or topology” comprises the 3D features relating to or comprising a vessel or one or more networks of vessels, which can be configured to facilitate or transport media (e.g., vasculature of a biological system). In some cases, media may comprise but are not limited to blood, bile, urine, air, or oxygen. Moreover, as understood by a person of ordinary skill in the art, the term “vasculature” refers to a perfusable 2D or 3D interconnected tubular transport system. More particularly, a “vasculature” or “vascular structure” can convey biological fluids, nutrients, drugs, biologics, and/or gases or other substances to a cell, cellular aggregate, or tissue, including to maintain viability of the cell/tissue. “Vasculature” or a “vasculature structure” may also convey waste away from a cell, cellular aggregate, or tissue. In some cases, “vasculature” or a “vascular structure” described herein interpenetrates or is embedded within another network or within a tissue or a tissue mimic or substitute or extracellular matrix. Further, in some embodiments, the “vasculature” or “vascular structure” of a system, or method described herein does not consist of or comprise regular cylinders or other regular geometric shapes (e.g., such regular geometric shapes joined together to form a network), but instead has an irregular geometric structure, shape, or cross-section. Additionally, in some cases, the “vasculature” or “vascular structure” of a system or method described herein has a perfusable architecture or structure mimicking a mammalian (e.g., human) vascular system that includes an artery, arteriole, capillary bed, venule, vein, or a combination of two or more of the foregoing.

The terms “three-dimensional printing system,” “three-dimensional printer,” “printing,” and the like generally describe various solid freeform fabrication techniques for making three-dimensional articles or objects by stereolithography (SLA), digital light processing (DLP), selective deposition, jetting, fused deposition modeling (FDM), electric field-assisted additive manufacturing (EFA), multi-jet modeling (MJM) or multi-jet printing (MJP), and other additive manufacturing techniques now known in the art or that may be known in the future that use a build material to fabricate three-dimensional objects.

### I. Compositions for 3D Bioprinting

In one aspect, compositions or build materials for use with a 3D printer or additive manufacturing system are described herein. In some embodiments, a composition described herein comprises an aqueous build material and a photoinitiator. In some cases, the aqueous build material comprises one or more natural materials, such as silk, gelatin, collagen, and albumin. In some implementations, the silk is an aqueous silk fibroin. Additionally, in some embodiments, a composition described herein further comprises a photoacid. A composition described herein may also comprise a leaching agent, salt, and/or a light-absorbing additive material. Other components may also be present in some embodiments of compositions described herein.

Turning now in more detail to specific components of compositions described herein, a composition described herein comprises an aqueous build material. Build materials, also known as inks or photoinks, are liquid materials configured to form various 3D objects, articles, or parts via 3D printing or additive manufacturing systems. In some instances, a build material can be solid at ambient temperatures and convert to liquid at elevated printing temperatures. In other instances (e.g., in the case of aqueous build materials described herein), a build material is liquid at ambient temperatures. Moreover, in some cases, an aqueous build material described herein can be solidified using a laser or other source of electromagnetic radiation. Additionally, in some cases, an aqueous build material described herein can be solidified using an electrode system or other source of electric signal.

Solidification of the aqueous build material can be carried out in any manner not inconsistent with the objectives of the present disclosure. In some embodiments, for example, solidification comprises irradiating a composition comprising the aqueous build material with electromagnetic radiation having sufficient energy to generate sufficient transferable heat, or exposing the composition comprising the aqueous build material to a reactive species that can initiate solidification (e.g., a photoinitiator or other species that has already been “activated” to provide a reactive moiety such as a free-radical moiety). In some embodiments, for example, solidification comprises applying sufficient electric signal to a composition comprising the aqueous build material to solidify one or more locations of a bath containing the aqueous build material.

One non-limiting example of an aqueous build material described herein is an aqueous silk fibroin material. It is understood that silk is a protein formed by the silkworm, Bombyx mori, which is produced in threads and generated subject to a pH gradient in the silk gland of the silk worm. Within the silk gland, different regions consist of varying pH ranges: the posterior silk gland ranges from approximately pH 8.2 to 7.2, the middle silk gland is maintained at a pH of approximately 7.0, and the anterior silk gland ranges from approximately pH 6.8 to 6.2. It is at the posterior silk gland that fibroin synthesis occurs, while the middle silk gland is responsible for fibroin storage. Further, it is the anterior silk gland where the sol-gel transition of silk occurs, such that the silk solution is viscous at the extrusion apparatus of the silkworm. As described in the present disclosure, a pH gradient may be used to generate artificial or bioprinted silk materials, such as 3D networks.

Moreover, it is understood that the sol-gel transition point of fibroin solution (in terms of pH) is approximately pH 5.0-5.5, though the pH in Bombyx silk gland maintains a generally higher pH. Not intending to be bound by any particular theory, it is believed that this difference in pH for sol-gel transition indicates acidification within the silk gland. Herein, acidification is used in addition to heat transfer, in some instances, to initiate localized solidification of aqueous silk fibroin.

Aqueous silk fibroin is an FDA approved material, making it an attractive aqueous build material for generation of whole or partial 3D structures supporting silk organs (where “FDA” refers to the U.S. Food and Drug Administration). Further, silk has desirable strength properties, such that compositions comprising aqueous silk fibroin that are solidified in layers according to methods disclosed herein may include interlaminations that hybridize nearly seamlessly. However, other examples of aqueous build materials include, but are not limited to, collagen (such as type I collagen, type II collagen, or type III collagen), fibrin, chitosan, alginate, albumin, oxidized alginate, starch, hyaluronic acid, laminin, agarose, gelatin, glucan, elastin, and combinations thereof.

In general, the aqueous build material component of a composition described herein can be present in the composition in any amount not inconsistent with the technical objectives of the present disclosure. In some preferred embodiments, for example, the aqueous build material is aqueous silk fibroin and is present in an amount or concentration of 5-15 weight percent (wt. %), based on total weight of the composition. In some instances, the aqueous silk fibroin component (or other aqueous build material component described in the preceding paragraph) is present in an amount of 0.1-30 wt. %, 0.1-20 wt. %, 0.1-15 wt. %, 0.1-10 wt. %, 0.1-5 wt. %, 1-30 wt. %, 1-20 wt. %, 1-15 wt. %, 1-10 wt. %, 1-5 wt. %, 5-30 wt. %, 5-20 wt. %, 5-15 wt. %, 5-10 wt. %, 10-30 wt. %, 10-20 wt. %, 10-15 wt. %, 15-30 wt. %, 15-20 wt. %, 20-30 wt. %, based on the total weight of the composition.

A composition described herein may also comprise a combination of silk fibroin and one or more additional build materials, such as collagen (such as type I collagen, type II collagen, or type III collagen), fibrin, chitosan, alginate, albumin, oxidized alginate, starch, hyaluronic acid, laminin, agarose, gelatin, glucan, and/or elastin.

As stated above, compositions described herein can comprise a photoinitiator. Any photoinitiator not inconsistent with the objectives of the present disclosure may be used in a composition described herein. The photoinitiator of a composition having an aqueous build material described herein is operable to initiate solidification of the aqueous build material when the photoinitiator is exposed to incident radiation having a Gaussian distribution of wavelengths and a peak wavelength 2. Moreover, the aqueous build material composition has a penetration depth (Dp) and a critical energy (Ec) at the wavelength 2. The terms Dp and E, are described in further detail below.

In some embodiments, for example, the photoinitiator component comprises an alpha-cleavage type (unimolecular decomposition process) photoinitiator or a hydrogen abstraction photosensitizer-tertiary amine synergist, operable to absorb light between about 250 nm and about 400 nm, between about 250 nm and 405 nm, or between about 300 nm and about 385 nm, to yield free radical(s). Examples of alpha cleavage photoinitiators are Irgacure 184 (CAS 947-19-3), Irgacure 369 (CAS 119313-12-1), Irgacure 819 (CAS 162881-26-7), and Irgacure 2959 (CAS 106797-53-9). An example of a photosensitizer-amine combination is Darocur BP (CAS 119-61-9) with diethylaminoethylmethacrylate.

In addition, in some instances, photoinitiators comprise benzoins, including benzoin, benzoin ethers, such as benzoin methyl ether, benzoin ethyl ether and benzoin isopropyl ether, benzoin phenyl ether and benzoin acetate, acetophenones, including acetophenone, 2,2-dimethoxyacetophenone and 1,1-dichloroacetophenone, benzil, benzil ketals, such as benzil dimethyl ketal and benzil diethyl ketal, anthraquinones, including 2-methylanthraquinone, 2-ethylanthraquinone, 2-tert-butylanthraquinone, 1-chloroanthraquinone and 2-amylanthraquinone, triphenylphosphine, benzoylphosphine oxides, such as 2,4,6-trimethylbenzoyldiphenylphosphine oxide (Lucirin TPO), benzophenones, such as benzophenone and 4,4′-bis(N,N′-dimethylamino)benzophenone, thioxanthones and xanthones, acridine derivatives, phenazine derivatives, quinoxaline derivatives or 1-phenyl-1,2-propanedione, 2-O-benzoyl oxime, 1-aminophenyl ketones or 1-hydroxyphenyl ketones, such as 1-hydroxycyclohexyl phenyl ketone, phenyl 1-hydroxyisopropyl ketone and 4-isopropylphenyl 1-hydroxyisopropyl ketone.

Suitable photoinitiators can also comprise photoinitiators operable for use with a HeCd laser radiation source, including acetophenones, 2,2-dialkoxybenzophenones and 1-hydroxyphenyl ketones, such as 1-hydroxycyclohexyl phenyl ketone or 2-hydroxyisopropyl phenyl ketone (=2-hydroxy-2,2-dimethylacetophenone). Additionally, in some cases, suitable photoinitiators comprise those operable for use with an Ar laser radiation source including benzil ketals, such as benzil dimethyl ketal. In some embodiments, a suitable photoinitiator comprises an a-hydroxyphenyl ketone, benzil dimethyl ketal or 2,4,6-trimethylbenzoyldiphenylphosphine oxide or a mixture thereof.

Another class of photoinitiators that may be included in a composition described herein comprises ionic dye-counter ion compounds capable of absorbing actinic radiation and generating free radicals for polymerization initiation. In some embodiments, composition containing ionic dye-counter ion compounds can be polymerized upon exposure to visible light within the adjustable wavelength range of about 400 nm to about 700 nm. Ionic dye-counter ion compounds and their mode of operation are disclosed in EP-A-0 223 587 and U.S. Pat. Nos. 4,751,102; 4,772,530; and 4,772,541.

Other suitable photointiators include Eosin-Y, which is a type II initiator requiring a second molecule, such as an electron donor, to initiation polymerization. Additional suitable photoinitiators include lithium acylphosphinate, tris(triphenlphosphine) ruthenium (II), and camphorquinone.

A photoinitiator component can be present in a composition described herein in any amount not inconsistent with the objectives of the present disclosure. In some embodiments, a photoinitiator component is present in a composition in an amount of up to about 7 wt. %, up to about 5 wt. %, up to about 3 wt. %, or up to about 2 wt. %, based on the total weight of the composition. In some cases, a photoinitiator is present in an amount of about 0.1-7 wt. %, 0.1-5 wt. %, 0.1-3 wt. %, 0.1-2 wt. %, 0.5-5 wt. %, 0.5-3 wt. %, 0.5-2 wt. %, 1-7 wt. %, 1-5 wt. %, or 1-3 wt. %, based on the total weight of the composition. In some especially preferred embodiments, a composition described herein comprises a photoinitiator component in an amount of up to about 5 wt. %. For example, in some instances, the photoinitiator component is present in the composition in an amount of 0.1-5 wt. % or 0.5-5 wt. % or, even more preferably, 1-5 wt. %, 1-3 wt. %, or 2-4 wt. %, based on the total weight of the composition.

It is further to be understood that the amounts (weight percents) described in the immediately preceding paragraph refer to photoinitiators that are non-oligomeric and non-polymeric. That is, the amounts described above refer to “monomeric” or “molecular” photoinitiators, which may, for instance, have a molecular weight of less than 400. However, it is also to be understood that oligomeric or polymeric photoinitiators may be used in compositions and methods described herein. But in such an instance (when an oligomeric or polymeric photoinitiator is used), then the amounts (weight percents) above are to be calculated without taking into account the weight of the oligomeric or polymeric portion or moiety of the oligomeric or polymeric photoinitiator. In other words, to determine the overall amount (weight percent) of the oligomeric or polymeric photoinitiator that is present in the composition, the calculation (specifically, the numerator) should be based on only the molecular weight of the photoactive moiety of the photoinitiator, not on the molecular weight(s) of the remaining moieties or repeating units of the oligomeric or polymeric photoinitiator (for purposes of the present disclosure).

A composition described herein may additionally include a photoacid component for acidifying the composition upon exposure to electromagnetic radiation. Any photoacid not inconsistent with the objectives of the present disclosure may be used in a composition described herein. For example, in some instances, the photoacid is a pyranine photoacid (or 8-hydroxypyrene-1,3,6-trisulfonic acid, HPTS) that exhibits a change in fluorescence, such that a fluorescence signature after exposure to electromagnetic radiation indicates local acidification. Pyranine has pKₐ values of approximately 7.4 and 0.4 for ground and excited states, respectively, and the protonated and deprotonated states absorb and emit at different wavelengths, allowing pyranine to function as a fluorescent pH indicator. However, other photoacids, whether they have a pH indicating function or not, may be used in a composition described herein if not inconsistent with the objectives of the present disclosure. Other non-limiting examples of photoacids that may be used in some embodiments described herein include merocyanine photoacids, indazole photoacids, or triarylsulfonium salt photoacids.

A photoacid of the present disclosure is configured to acidify the composition comprising the aqueous build material when electromagnetic radiation is applied. Upon acidification (alone or in combination with heat generated by light or an electric current, or in combination with another solidification stimulus described herein), the aqueous build material solidifies and forms desired networks with defined 3D architecture. It is further to be understood that, in some embodiments, the use of a photoacid that undergoes proton disassociation reversibly is preferred, as compared to the use of a chemical species that undergoes proton disassociation irreversibly in response to irradiation with light. Such species that undergo irreversible proton disassociation can be referred to as photoacid generators (PAGs) rather than photoacids (PAHs), in some cases. In some embodiments described herein, a PAG is not used in the composition, or is used in an amount or concentration of less than 0.1 M (e.g., 0.01 M or less, 0.001 M or less, or 0.0001 M or less).

In general, the photoacid component of a composition described herein can be present in the composition in any amount not inconsistent with the technical objectives of the present disclosure. In some embodiments, for example, the photoacid component is present in an amount or concentration of 1 M or less. In some instances, the photoacid component is present in an amount (e.g., a non-zero amount) of 0.5 M or less, 0.2 M or less, 0.1 M or less, 0.05 M or less, or 0.01 M or less. In some cases, the photoacid component is present in an amount or concentration of 0.1-1 M, 0.1-0.5 M, 0.1-0.3 M, 0.2-1 M, 0.2-0.5 M, or 0.5-1 M.

A composition described herein may also include a leaching agent component for removing species, such as toxic species, from the solidified aqueous build material. Any leaching agent not inconsistent with the objectives of the present disclosure may be used in a composition described herein. In some instances, the leaching agent comprises tetramethylethylenediamine (TEMED), poly(ethylene glycol) (PEG), or poly(ethylene glycol) diacrylate (PEGDA). PEGDA can comprise a single poly(ethylene glycol) diacrylate species or multiple poly(ethylene glycol) diacrylate species of differing molecular weights. In some embodiments, a species of PEGDA have a weight average molecular weight of 0.1 kiloDalton (kDa) to 50 kDa or approximately 35 kDa. Any combination or mixture of poly(ethylene glycol) diacrylates of differing molecular weights is contemplated.

Compositions described herein, in some cases, also comprise a salt. Any salt not inconsistent with the technical objectives of the present disclosure may be used with the aqueous build material. In some instances, a salt may serve as a buffer in the composition. For instance, where the composition includes a photoacid and aqueous build material, a strongly buffered solution can be used with a high concentration of the photoacid and base. In general, the salt component of a composition described herein can be present in the composition in any amount not inconsistent with the technical objectives of the present disclosure. In some instances, the salt or buffer component is present in an amount or concentration of 0.01-2 M, 0.01-1.5 M, 0.01-1 M, 0.01-0.5 M, 0.01-0.3 M, 0.01-0.1 M, 0.05-2 M, 0.05-1.5 M, 0.05-1 M, 0.05-0.5 M, 0.05-0.3 M, 0.05-0.1 M, 0.1-2 M, 0.1-1.5 M, 0.1-1 M, 0.1-0.5 M, or 0.1-0.3 M.

Moreover, in some cases, a composition described herein comprises biocompatible, light-absorbing additive material configured to control light penetration. It is to be understood that the amounts of photoinitiator and/or light-absorbing additive material included in a composition described herein can be selected to obtain a desired Dp, Ec, and/or Dpr value, in combination with other components of the composition.

Additionally, it is to be understood that the parameters or properties Dp, Ec, and DPT are structural parameters or properties of the composition described herein. A discussion of the “structural” or “compositional” nature of these values can be found, for instance, in Chapter 4 of Paul F. Jacobs, Rapid Prototyping & Manufacturing: Fundamentals of Stereolithography (Society of Manufacturing Engineers, McGraw-Hill, 1992) (first edition) (hereinafter referred to as “Jacobs”). As understood by one of ordinary skill in the art, the value Dp is the penetration depth of the composition, defined as that depth of the composition which results in a reduction of the irradiance to a level equal to 1/e of the surface irradiance, where e is the base of natural logarithms (equal to 2.7182818 . . . ). Ec is the critical energy, which is the energy needed to obtain the gel point of a composition, as described on page 86 of Jacobs. Moreover, as further described by Jacobs (pages 86-89), the metric Ec is equal to the intercept of a working curve corresponding to a semilog plot of cure depth on the ordinate and the logarithm of maximum radiation exposure on the abscissa. Ec is assigned to the intercept, at which the cure depth is zero. The composition has a print through depth (Dpr), where Dpr refers to the total cure depth minus layer thickness.

A biocompatible, “light-absorbing additive material,” for reference purposes herein, is a material or chemical species that is not curable or substantially curable by the curing radiation described herein and that absorbs at least a portion of the radiation, without causing substantial curing of other components of the composition. Thus, a “light-absorbing” absorber material can also be referred to as a “non-curing” or “non-reactive” absorber material. Moreover, a light-absorbing additive material described herein that is not “substantially” curable or that does not cause “substantial” curing is understood to convert (or use) less than 5%, less than 1%, less than 0.5%, or less than 0.1% of absorbed radiation photons into (or in) a curing event. For example, a light-absorbing additive material described herein, in some embodiments, can convert less than 2%, less than 1%, less than 0.5%, or less than 0.1% of absorbed photons into a free-radical species that can initiate or participate in polymerization or another curing process.

It is further to be understood that a light-absorbing additive material described herein can be a polymerization “spectator” (i.e., non-polymerizing or non-polymerization-initiating) species that nevertheless “competes” with a photoinitiator (or photoacid) of the composition for absorption of photons of incident curing radiation. Thus, in some cases, a light-absorbing additive material and a photoinitiator (or photoacid) of the composition described herein may have substantially overlapping photon absorption profiles, particularly in a region of the electromagnetic spectrum corresponding to or including the peak wavelength λ described above.

However, it is to be understood that a light-absorbing additive material and a photoinitiator (or photoacid) of a composition described herein need not have the same absorbance, optical density, extenuation coefficient, and/or molar extinction coefficient at the wavelength λ or at any other specific wavelength. Instead, the light-absorbing additive material and the photoinitiator (or photoacid) can have differing absorbances, optical densities, extenuation coefficients, and/or molar extinction coefficients at the wavelength 2, as well as at other wavelengths.

A composition described herein may also comprise water, including as a solvent or diluent. In some cases, water is present in a composition as the “balance” of the composition, or the amount needed to reach 100 wt. % of the composition, after taking into account the amounts of the remaining components of the composition. In some embodiments, for example, water is present in a composition described herein in an amount up to 90 wt. %, up to 85 wt. %, up to 80 wt. %, up to 75 wt. %, up to 70 wt. %, up to 60 wt. %, up to 50 wt. %, up to 40 wt. %, up to 35 wt. %, up to 30 wt. %, up to 25 wt. %, up to 20 wt. %, or up to 15 wt. %, based on the total weight of the composition.

Compositions or materials described herein can have a variety of properties in an aqueous or solidified state, including properties related to the microstructure of the composition or material, which may be a complex mixture or other complex material system. In some embodiments, such structural features or other properties relate to the composition or material in a solidified or polymerized state. A composition or material in a “solidified” or “polymerized” state, as used throughout the present disclosure, comprises a composition or material that includes a curable material or polymerizable or crosslinkable component that has been at least partially solidified. For instance, in some cases, a solidified composition or material is at least about 70% polymerized or cross-linked or at least about 80% polymerized or cross-linked. In some embodiments, a solidified composition or material is at least about 85%, at least about 90%, at least about 95%, at least about 98%, or at least 99% polymerized or cross-linked. In some instances, a solidified composition or material is between about 80% and about 99% polymerized or cross-linked. The degree of polymerization or cross-linking can be determined using any protocol or method not inconsistent with the technical objectives of the present disclosure. It is further to be understood that the degree of polymerization or cross-linking described herein is different than “degree of polymerization” defined as the number of repeating units in a polymer molecule.

In some embodiments, a composition described herein when solidified or polymerized has a biomechanical strength sufficient to maintain 3D network complexities.

Additionally, in some embodiments, the compositions described herein, when non-solidified, have a viscosity profile consistent with the requirements and parameters of one or more 3D printing systems, such as an MJP, SLA, or DLP system. For example, in some cases, a composition described herein has a dynamic viscosity at 23 or 30° C. of 1600 centipoise (cP) or less, 1200 cP or less, 800 cP or less, or 500 cP or less at 23 or 30° C., when measured according to ASTM standard D2983 (e.g., using a Brookfield Model DV-II+Viscometer). In some cases, a composition described herein when non-solidified exhibits a dynamic viscosity of about 200-1600 cP, about 200-1200 cP, about 200-800 cP, about 200-500 cP, or about 200-400 cP at 23 or 30° C., when measured according to ASTM D2983. Further, in some cases, a composition described herein, in an unsolidified state, has a dynamic viscosity of 8 cP to 14 cP at a temperature of 80° C., when determined according to ASTM D2983.

Compositions described herein can also include, have, or exhibit any combination of components and/or properties described hereinabove individually, provided that the combination of components and/or properties is not inconsistent with the principles and technical objectives of the present invention. Moreover, in some embodiments, compositions described herein have a combination of compositional characteristics that can be especially preferred for providing biocompatibility, feature resolution, and/or strength.

Compositions described herein can be produced in any manner not inconsistent with the technical objectives of the present disclosure. In some embodiments, for instance, a method for the preparation of a composition described herein comprises the steps of mixing the components of the composition, optionally melting the mixture, and filtering the (optionally molten) mixture. In some cases, the components are mixed and optionally melted at a temperature between about 22° C. and about 35° C., or at a temperature in the range of 25-35° C., 25-55° C., 35-65° C., or 45-75° C. In some embodiments, a composition described herein is produced by placing all components of the composition in a reaction vessel, optionally heating the resulting mixture, and stirring the resulting mixture at a temperature between about 22° C. and about 75° C. The stirring (and optionally the heating) are continued until the mixture attains a substantially homogenized liquid (or molten) state. In general, the liquid (or molten) mixture can be filtered while in a flowable state to remove any large undesirable particles that may interfere with jetting or extrusion or other printing process. The filtered mixture can then be cooled to ambient temperatures (if cooling is needed) and stored until ready for use in a 3D printing system. In some instances, the filtered mixture can be stored at temperatures below ambient temperatures, such as 4° C. or below.

### II. Methods of Forming a 3D Network by Bioprinting of an Aqueous Build Material

In another aspect, methods of forming or “printing” a network, such as a vascular network, ductal network, airway network, neural network, or a combination of two or more of the foregoing, by additive manufacturing are described herein. Methods of forming a 3D article or network described herein can include forming the 3D article from a plurality of layers of a composition described herein in a layer-by-layer manner. In such cases, the composition can be used as a build material. Methods of forming a 3D article by additive manufacturing can also include forming the object in a manner other than a layer-by-layer manner. Any composition described hereinabove in Section I may be used in a method described herein. For example, in some embodiments, a method described herein comprises providing a composition, wherein the composition comprises an aqueous build material with or without a photoinitiator. Further, in some such cases, the compositions further comprises one or more salts, leaching agents, photoacids, and/or biocompatible light-absorbing additives.

In some cases, a method described herein comprises providing a bath comprising a composition described above, and selectively solidifying a portion of the bath using incident radiation having a Gaussian distribution of wavelengths and a peak wavelength at the wavelength 2. Moreover, in some embodiments described herein, the bath is selectively solidifying according to a digital file or image of the desired article, such as according to preselected computer aided design (CAD) parameters presenting a 3D model. Moreover, in some cases, one or more layers of a build material described herein has a thickness of about 10 μm to about 100 μm, about 10 μm to about 80 μm, about 10 μm to about 50 μm, about 10 μm to about 40 μm, about 20 μm to about 100 μm, about 20 μm to about 80 μm, or about 20 μm to about 40 μm. Other thicknesses are also possible.

In some embodiments, the bath comprising the composition is prepared at ambient temperature prior to application of incident radiation. In some cases, bath comprising the composition is prepared at a temperature below ambient temperature prior to application of incident radiation, such as at or below 4° C.

Performing a printing process described herein can provide a printed 3D article or network from a bath described herein that has a high feature resolution. The “feature resolution” of an article, for reference purposes herein, can be the smallest controllable physical feature size of the article or the pixel or voxel size of the printing process, where it is understood that “pixel” and “voxel” refer to the CAD parameter or other digital model of the article. In some embodiments, a printed article described herein has an average voxel size greater than 50 μm per side on average (e.g., when the average voxel size corresponds to a volume having an average length in all three dimensions of 50-100 μm, 50-75 μm, 60-100 μm, 60-80 μm, or 60-70 μm). In other cases, a printed article described herein has an average voxel size of less than 50 μm, less than 40 μm, less than 30 μm, or less than 20 μm per side on average (e.g., when the average voxel size corresponds to a volume having an average length in all three dimensions of 10-45 μm, 10-40 μm, 10-30 μm, 10-25 μm, 10-20 μm, 15-45 μm, or 15-40 μm).

Additionally, it is to be understood that methods of printing a 3D article described herein can include, for example, MJP, DLP, or SLA 3D printing methods. For example, a method described herein can comprise solidifying the layers or portions of the bath, including with radiation or electric signal described above. Moreover, solidifying can comprise polymerizing one or more polymerizable moieties or functional groups of one or more components of the bath. In some cases, a first portion of the bath is solidified prior to the solidification of a second portion of the bath, which may or may not be adjacent to the first portion. Additionally, solidifying one or more portions of the bath, in some embodiments, is carried out by exposing the one or more portions or locations within each portion to electromagnetic radiation, such as UV light, visible light, or infrared light, as described above, or by exposing the one or more portions or locations within each portion to electric signal, such as an electromagnetic field.

It should further be noted that a wavelength 2 used to solidify a material according to a method described herein can be any wavelength not inconsistent with the objectives of the present disclosure. For example, in some cases, A is a wavelength in the ultraviolet (UV) or visible region of the electromagnetic spectrum. In some cases, the peak wavelength λ is in the infrared (IR) region of the electromagnetic spectrum. In some embodiments, the wavelength A is between 250 nm and 400 nm, between 300 nm and 385 nm, or between 385 nm and 405 nm. In other cases, the wavelength λ is between 600 nm and 800 nm or between 900 nm and 1.3 μm. However, the precise wavelength λ is not particularly limited.

Further details regarding various methods, including “vat polymerization” or “vat solidification” methods (such as SLA or DLP) and “electric field assisted” methods (EFA additive manufacturing), are provided below.

### A. Vat Polymerization Methods

It is possible to form a 3D article from a composition comprising the aqueous build material described herein using a vat polymerization or solidification method, such as an SLA method. Thus, in some cases, a method of printing a 3D article or network described herein comprises retaining a build material, which may be any composition described in Section I above, described herein in a fluid state in a container, such as a bath, and selectively applying energy (particularly, for instance, an illumination beam or projection from an optical system, such as an optical system of an SLA system or DLP system) to the build material in the container to solidify at least one or more locations of a first bath portion of a fluid layer of the build material, thereby forming a solidified layer that defines a cross-section of the 3D article. Additionally, a method described herein can further comprise raising or lowering the solidified layer of build material via elevator system or other system configured to raise or lower the build plane. This elevating of the build plane provides a new or second bath portion of a fluid layer of unsolidified build material at the surface of the aqueous build material in the container, followed by again selectively applying energy (e.g., the illumination beam or projection) to the build material in the container to solidify at one or more locations of the new or second fluid layer of the build material to form a second solidified layer that defines a second cross-section of the 3D article. Further, the first and second cross-sections of the 3D article can be bonded or adhered to one another in the z-direction (or build direction corresponding to the direction of raising or lowering recited above) by the application of the energy for solidifying the build material.

Moreover, in some instances, the electromagnetic radiation has an average wavelength of 300-900 nm, and in other embodiments the electromagnetic radiation has an average wavelength that is less than 300 nm. In some cases, the radiation is provided by a computer controlled illumination beam, laser beam, light projector, digital micromirror device (DMD), or other light source.

In some cases, the direction of electromagnetic radiation towards one or more locations of the container of build material generates transferable heat, which in some cases may alone or in combination with one or more polymerizing moieties result in local solidification. For instance, where the composition comprises an aqueous build material without a photoinitiator or photoacid, solidification may occur based on heat transfer from the electromagnetic radiation. For example, aqueous build materials such as albumin or aqueous silk fibroin may be solidified using transferred heat without other composition components, such as photoinitiators or photoacids. It is understood in such situations, however, that diffusion may be high (that is, printing resolution may be low), unless temperature is highly confined or controlled spatially. As such, cryogenic bioprinting conditions may be used to address high thermal diffusion in such instances. In some cases, reduction in temperature of the 3D printing environment and/or composition for 3D printing may serve to rapidly quench heat from the photosolidification process, confining the solidification to desired regions of the container holding aqueous build materials. In yet other cases, in addition to or in alternative to cooling, resolution from heat transfer-based solidification may be improved by adjustment of laser or other optical power to reduce diffusion.

In another example, where the composition comprises an aqueous build material and a photoacid, solidification may occur based on energy transfer from the electromagnetic radiation causing the photoacid to acidify the composition in the container at one or more locations, as desired. This acidification prompts the precipitation or non-covalent polymerization of the aqueous build material at the one or more locations, which, along with or in addition to heat transfer, generates solidified build material at those locations.

A method described herein can also comprise planarizing a new layer of aqueous build material provided by raising or lowering an elevator platform. Such planarization can be carried out, in some cases, by a wiper or roller.

It is further to be understood that the foregoing process can be repeated a desired number of times to provide the 3D article. For example, in some cases, this process can be repeated “n” number of times, wherein n can be up to about 100,000, up to about 50,000, up to about 10,000, up to about 5000, up to about 1000, or up to about 500. Thus, in some embodiments, a method of printing a 3D article described herein can comprise selectively applying energy to a build material in a container to solidify at least a portion of an nth fluid layer of the build material, thereby forming an nth solidified layer that defines an nth cross-section of the 3D article, raising or lowering the nth solidified layer of build material to provide an (n+1)th layer of unsolidified build material at the surface of the fluid build material in the container, selectively applying energy to the (n+1)th layer of build material in the container to solidify at least a portion of the (n+1)th layer of the build material to form an (n+1)th solidified layer that defines an (n+1)th cross-section of the 3D article, raising or lowering the (n+1)th solidified layer of build material to provide an (n+2)th layer of unsolidified build material at the surface of the fluid build material in the container, and continuing to repeat the foregoing steps to form the 3D article. Further, it is to be understood that one or more steps of a method described herein, such as a step of selectively applying energy to a layer of build material, can be carried out according to an image of the 3D article in a computer-readable format. General methods of 3D printing using stereolithography are further described, inter alia, in U.S. Pat. Nos. 5,904,889 and 6,558,606.

In a vat polymerization or solidification method such as described above, the build material may be partially solidified. A “partially solidified” build material, for reference purposes herein, is one that can undergo further solidification. For example, a partially solidified build material is up to about 30% polymerized or cross-linked or up to about 50% polymerized or cross-linked. In some embodiments, a partially solidified build material is up to about 60%, up to about 70%, up to about 80%, up to about 90%, or up to about 95% polymerized or cross-linked.

For example, in some embodiments, selectively applying energy to the build material in the container to solidify at least a portion of a fluid layer of the build material may include partially solidifying at least a portion of a fluid layer of the build material. In other embodiments, partial solidifying of at least a portion of a fluid layer of the build material may occur after a first layer of the build material is provided and solidified, before or after a second layer of the build material is provided or solidified, or before or after one, several, or all subsequent layers of the build material are provided or solidified.

Additionally, in some embodiments of a vat polymerization or solidification method described herein, after partial solidifying or after the desired 3D article is formed, post-solidifying may be performed. For example, in some cases, post-solidifying is carried out after all layers of the build material are provided or solidified to form a desired 3D article, after partially solidifying all layers of the build material, or after both of the foregoing steps have been performed. Moreover, in some embodiments, post-solidifying comprises photosolidification, including with electromagnetic radiation described. Again, any electromagnetic radiation source not inconsistent with the objectives of the present disclosure may be used for a post-solidifying step described herein. For example, in some embodiments, the electromagnetic radiation source can be a light source that has a higher energy, a lower energy, or the same energy as the electromagnetic radiation source used for partial solidifying. In some cases wherein the electromagnetic radiation source used for post-solidifying has a higher energy (i.e., a shorter wavelength) than that used for partial solidifying, a Xe arc lamp can be used for partial solidifying and a Hg lamp can be used for post-solidifying.

The desired 3D article may be, for example, an article that corresponds to the design in a CAD file or other digital file, image, or model corresponding to the desired 3D article.

### B. Electric Field-Assisted Additive Manufacturing (EFA)

It is also possible to form a 3D article from a composition comprising an aqueous build material and a salt described herein using an electric field-assisted (EFA) method. Thus, in some cases, a method of printing a 3D article or network described herein comprises retaining a build material, which may be any composition described in Section I above, in a fluid state in a container, such as a bath, and selectively applying an electric signal (particularly, for instance, an electric field from a pair of electrodes of an electrode system) to the build material in the container to solidify at least one or more locations of a first bath portion of a fluid layer of the build material, thereby forming a solidified layer that defines a cross-section of the 3D article.

Additionally, a method described herein can further comprise raising or lowering the solidified layer of build material via elevator system or other system configured to raise or lower the build plane. This elevating of the build plane provides a new or second bath portion of a fluid layer of unsolidified build material at the surface of the aqueous build material in the container, followed by again selectively applying electric signal to the build material in the container to solidify at one or more locations of the new or second fluid layer of the build material to form a second solidified layer that defines a second cross-section of the 3D article. Further, the first and second cross-sections of the 3D article can be bonded or adhered to one another in the z-direction (or build direction corresponding to the direction of raising or lowering recited above) by the application of the electric signal for solidifying the build material.

In some instances, the electric signal includes an electric, magnetic, or acoustic field. The direction of electric signal along one or more locations of the container of build material generates transferable heat, which in some cases may alone or in combination with one or more polymerizing moieties result in local solidification. In some cases, the electric field is provided by a computer controlled electrode system or other electric signal source. The desired 3D article may be, for example, an article that corresponds to the design in a CAD file or other digital file, image, or model corresponding to the desired 3D article.

As with the methods of Section IIA above, the foregoing process can be repeated a desired number of times to provide the 3D article. Also similar to the methods of Section IIA, the 3D article may be partially solidified, and after partial solidifying or after the desired 3D article is formed, post-solidifying may be performed.

### III. Printed 3D Articles

In another aspect, printed 3D articles, networks, or fluid networks are described herein. In some embodiments, a printed 3D article, network, or fluid network is formed from a composition described herein and/or using a method of 3D printing described herein. Any composition described hereinabove in Section I may be used. For example, in some cases, the composition comprises an aqueous build material and a photoinitiator or photoacid. Further, in some such cases, the composition comprises one or more of a photoacid, a salt, a leaching agent, and/or a biocompatible light-absorbing additive. Similarly, any method described hereinabove in

Section IIA or IIB may be used to form a printed 3D article, network, or fluid network according to the present disclosure.

A composition and/or method according to the present disclosure can be used to form a variety of printed 3D articles, networks, or fluid networks by additive manufacturing, without particular limitation. Similarly, printed 3D articles, networks, or fluid networks printed according to methods described herein can find application in a variety of fields. However, in some preferred embodiments, the article is a network, such as a vascular network, ductal network, airway network, neural network, or a combination of two or more of the foregoing for use as a part or whole bioengineered tissue or organ. Other articles can also be formed by a method of additive manufacturing described herein.

### EXAMPLES

### Preparation of Compositions

Build materials according to some embodiments described herein may be prepared as follows. Specifically, to prepare various build materials, the components in the following Tables 1-3 may be mixed in a reaction vessel to form specific build materials, as identified hereinbelow. The amounts of various components in Tables 1-3 refer to the wt. % of each component, based on the total weight of the composition, or the concentration, as indicated. Dashes (--) indicate that the specific component was absent. It is to be understood that the balance of each exemplary build material is water. For each build material, the appropriate mixture may be heated to a temperature of about 22-85° C. with stirring. The heating and stirring may be continued until the mixture attains a substantially homogenized molten state. The molten mixture may be optionally filtered. The filtered mixture may then be allowed to cool to ambient temperature.

TABLE 1
Build Material Compositions.

| | Build | Build | Build | Build | Build | Build |
|---|---|---|---|---|---|---|
| | Material | Material | Material | Material | Material | Material |
| | 1 | 2 | 3 | 4 | 5 | 6 |
| Aqueous Build | 5-15 wt. % | ≥5 wt. % | ≥5 wt. % | ≥5 wt. % | ≥5 wt. % | ≥5 wt. % |
| Material | | | | | | |
| Photoacid | 1M or | 1M or | 1M or | — | — | — |
| | less | less | less | | | |
| Photoinitiator | 5 wt. % or | 5 wt. % or | — | — | 5 wt. % or | 5 wt. % or |
| | less | less | | | less | less |
| Leaching | 1-40 wt. % | — | — | — | — | 1-40 wt. % |
| Agent | | | | | | |

TABLE 2
Build Material Compositions.

| | Build | Build | Build | Build | Build | Build |
|---|---|---|---|---|---|---|
| | Material | Material | Material | Material | Material | Material |
| | 7 | 8 | 9 | 10 | 11 | 12 |
| Aqueous Build | 10 wt. % | 15 wt. % | 20 wt. % | 20 wt. % | 25 wt. % | 30 wt. % |
| Material | | | | | | |
| Photoacid | 0.1M | 0.3M | 0.5M | 0.5M | 0.7M | 1M |
| Photoinitiator | — | 3 wt. % | — | — | — | 2 wt. % |
| Leaching | 3 wt. % | 10 wt. % | 5 wt. % | 7 wt. % | — | 12 wt. % |
| Agent | | | | | | |

TABLE 3
Build Material Compositions.

| | Build | Build | Build | Build | Build | Build |
|---|---|---|---|---|---|---|
| | Material | Material | Material | Material | Material | Material |
| | 13 | 14 | 15 | 16 | 17 | 18 |
| Aqueous Build | 10 wt. % | 15 wt. % | 20 wt. % | 20 wt. % | 25 wt. % | 30 wt. % |
| Material | | | | | | |
| Photoacid | 0.1M | 0.3M | — | 0.5M | 0.7M | — |
| Salt | — | — | 5 wt. % | 5 wt. % | — | 10 wt. % |
| Photoinitiator | — | 3 wt. % | — | — | — | 2 wt. % |
| Leaching | 3 wt. % | 10 wt. % | 5 wt. % | 7 wt. % | — | 12 wt. % |
| Agent | | | | | | |

In Tables 1-3, the aqueous build material is silk fibroin; the photoacid is pyranine; the leaching agent is PEG/PEGDA; and the salt is phosphate buffered saline (PBS). While exemplary build material compositions are described in Tables 1-3, other compositions with these components, a portion of these components, or other components are contemplated.

Some additional non-limiting example Embodiments are as follows.

Embodiment 1. A method of making a network, the method comprising:
providing a bath comprising aqueous build material;
providing an optical system configured to generate an illumination beam or projection; and directing the illumination beam or projection towards the bath to solidify the aqueous build material at one or more locations of the bath,
wherein the solidified aqueous build material forms the network.

Embodiment 2. The method of Embodiment 1, wherein the network comprises a vascular network, ductal network, airway network, neural network, or a combination of two or more of the foregoing.

Embodiment 3. The method of Embodiment 1 or Embodiment 2, wherein the illumination beam or projection is directed towards the bath based on a 3D model of the network.

Embodiment 4. The method of any of Embodiments 1-3, further comprising:
providing a build plane holding a first portion of the bath, wherein the first portion includes the one or more locations, wherein directing the illumination beam or projection towards the bath to solidify the aqueous build material at the one or more locations comprises directing the illumination beam or projection at the first portion of the bath;
providing an elevator system configured to elevate the build plane to hold one or more different portions of the bath;
elevating the build plane to hold a second portion of the bath; and
directing the illumination beam or projection towards the second portion of the bath to solidify the aqueous build material at one or more second locations of the bath.

Embodiment 5. The method of any of the preceding Embodiments, wherein the aqueous build material comprises aqueous silk fibroin.

Embodiment 6. The method of Embodiment 5, wherein the aqueous silk fibroin is present in the bath in an amount of 5-15 weight percent, based on a total weight of the bath.

Embodiment 7. The method of any of the preceding Embodiments, wherein the bath further comprises a photoacid.

Embodiment 8. The method of Embodiment 7, wherein directing the illumination beam or projection towards the bath to solidify the aqueous build material comprises: causing, via energy transferred from the illumination beam or projection, the photoacid in the one or more locations to acidify the bath; and causing the acidified bath to solidify the aqueous build material at the one or more locations.

Embodiment 9. The method of Embodiment 7 or Embodiment 8, wherein the photoacid is present in the bath at a concentration of 1 M or less.

Embodiment 10. The method of Embodiment 7 or Embodiment 8, wherein the photoacid is present in the bath at a concentration of up to 0.1 M.

Embodiment 11. The method of any of the preceding Embodiments, wherein the bath is at a temperature of 4° C. or below.

Embodiment 12. The method of any of the preceding Embodiments, wherein the bath further comprises a leaching agent.

Embodiment 13. The method of Embodiment 12, wherein the leaching agent comprises one or more of TEMED, PEG, and PEGDA.

Embodiment 14. The method of any of the preceding Embodiments, wherein:
the bath further comprises a photoinitiator; and
the photoinitiator comprises one or more of a lithium acylphosphinate, Irgacure 2959, Eosin Y system, tris(triphenlphosphine) ruthenium (II), and camphorquinone.

Embodiment 15. The method of any of the preceding Embodiments, wherein the bath further comprises a biocompatible, light-absorbing additive material configured to control light penetration.

Embodiment 16. A method of generating a network, the method comprising:
providing a bath comprising aqueous build material and salt;
providing an electrode system comprising a pair of electrodes configured to generate an electric signal; and
applying the electric signal along an area of the bath to solidify the aqueous build material at one or more locations of the bath,
wherein the solidified aqueous build material forms the network.

Embodiment 17. The method of Embodiment 16, wherein the network comprises a vascular network, ductal network, airway network, neural network, or a combination of two or more of the foregoing.

Embodiment 18. The method of Embodiment 16 or Embodiment 17, wherein the electrical signal is directed along the area of the bath based on a 3D model of the network.

Embodiment 19. The method of any of Embodiments 16-18, further comprising:
providing a build plane holding a first portion of the bath, wherein the first portion includes the one or more locations, wherein applying the electrical signal along the area of the bath to solidify the aqueous build material at the one or more locations comprises applying the electrical signal along an area in the first portion of the bath;
providing an elevator system configured to elevate the build plane to hold one or more different portions of the bath;
elevating the build plane to hold a second portion of the bath; and
applying the electrical signal along an area in the second portion of the bath to solidify the aqueous build material at one or more second locations of the bath.

Embodiment 20. The method of any of Embodiments 16-19, wherein the aqueous build material comprises aqueous silk fibroin.

Embodiment 21. The method of Embodiment 20, wherein the aqueous silk fibroin is present in the bath in an amount of 5-15 weight percent, based on a total weight of the bath.

Embodiment 22. The method of any of Embodiments 16-21, wherein the bath further comprises a leaching agent.

Embodiment 23. The method of Embodiment 22, wherein the leaching agent comprises one or more of TEMED, PEG, and PEGDA.

Embodiment 24. A composition comprising:
an aqueous build material;
a photoinitiator; and
a photoacid.

Embodiment 25. The composition of Embodiment 24, wherein the aqueous build material comprises aqueous silk fibroin.

Embodiment 26. The composition of Embodiment 25, wherein the aqueous silk fibroin is present in the composition in an amount of 5-15 weight percent, based on a total weight of the composition.

Embodiment 27. The composition of Embodiment 24 or Embodiment 25, wherein the photoacid is present in the composition at a concentration of 1 M or less.

Embodiment 28. The composition of Embodiment 24 or Embodiment 25, wherein the photoacid is present in the composition at a concentration of 0.1 M or less.

Embodiment 29. The composition of any of Embodiments 24-28, further comprising a leaching agent.

Embodiment 30. The composition of Embodiment 29, wherein the leaching agent comprises one or more of TEMED, PEG, or PEGDA.

Embodiment 31. The composition of any of Embodiments 24-30, further comprising a salt.

Embodiment 32. The composition of any of Embodiments 24-32, wherein the photoinitiator comprises one or more of a lithium acylphosphinate, Irgacure 2959, Eosin Y system, tris(triphenlphosphine) ruthenium (II), or camphorquinone.

Embodiment 33. The composition of any of Embodiments 24-32, further comprising a biocompatible, light-absorbing additive material configured to control light penetration.

All patent documents referred to herein are incorporated by reference in their entireties. Various embodiments of the invention have been described in fulfillment of the various objectives of the invention. It should be recognized that these embodiments are merely illustrative of the principles of the present invention. Numerous modifications and adaptations thereof will be readily apparent to those skilled in the art without departing from the spirit and scope of the invention.

1. A method of making a network, the method comprising:
providing a bath comprising aqueous build material;
providing an optical system configured to generate an illumination beam or projection; and
directing the illumination beam or projection towards the bath to solidify the aqueous build material at one or more locations of the bath,
wherein the solidified aqueous build material forms the network, and
wherein the aqueous build material comprises aqueous silk fibroin.

2. The method of claim 1, wherein the network comprises a vascular network, ductal network, airway network, neural network, or a combination of two or more of the foregoing.

3. The method of claim 1, wherein the illumination beam or projection is directed towards the bath based on a 3D model of the network.

4. The method of claim 1, further comprising:
providing a build plane holding a first portion of the bath, wherein the first portion includes the one or more locations, wherein directing the illumination beam or projection towards the bath to solidify the aqueous build material at the one or more locations comprises directing the illumination beam or projection at the first portion of the bath;
providing an elevator system configured to elevate the build plane to hold one or more different portions of the bath;
elevating the build plane to hold a second portion of the bath; and
directing the illumination beam or projection towards the second portion of the bath to solidify the aqueous build material at one or more second locations of the bath.

5. The method of claim 1, wherein the aqueous silk fibroin is present in the bath in an amount of 5-15 weight percent, based on a total weight of the bath.

6. The method of claim 1, wherein the bath further comprises a photoacid.

7. The method of claim 6, wherein directing the illumination beam or projection towards the bath to solidify the aqueous build material comprises:
causing, via energy transferred from the illumination beam or projection, the photoacid in the one or more locations to acidify the bath; and
causing the acidified bath to solidify the aqueous build material at the one or more locations.

8. The method of claim 7, wherein the photoacid is present in the bath at a concentration of 1 M or less.

9. The method of claim 1, wherein the bath is at a temperature of 4° C. or below.

10. The method of claim 1, wherein the bath is at a temperature of 20-35° C.

11. The method of claim 1, wherein the bath further comprises a leaching agent.

12. The method of claim 11, wherein the leaching agent comprises one or more of TEMED, PEG, and PEGDA.

13. The method of claim 1, wherein:
the bath further comprises a photoinitiator; and
the photoinitiator comprises one or more of a lithium acylphosphinate, Irgacure 2959, Eosin Y system, tris(triphenlphosphine) ruthenium (II), and camphorquinone.

14. The method of claim 1, wherein the bath further comprises a biocompatible, light-absorbing additive material configured to control light penetration.

15. A method of generating a network, the method comprising:
providing a bath comprising aqueous build material and salt;
providing an electrode system comprising a pair of electrodes configured to generate an electric signal; and
applying the electric signal along an area of the bath to solidify the aqueous build material at one or more locations of the bath,
wherein the solidified aqueous build material forms the network, and
wherein the aqueous build material comprises aqueous silk fibroin.

16. The method of claim 15, wherein the network comprises a vascular network, ductal network, airway network, neural network, or a combination of two or more of the foregoing.

17. The method of claim 15, wherein the electrical signal is directed along the area of the bath based on a 3D model of the network.

18. The method of claim 15, further comprising:
providing a build plane holding a first portion of the bath, wherein the first portion includes the one or more locations, wherein applying the electrical signal along the area of the bath to solidify the aqueous build material at the one or more locations comprises applying the electrical signal along an area in the first portion of the bath;
providing an elevator system configured to elevate the build plane to hold one or more different portions of the bath;
elevating the build plane to hold a second portion of the bath; and
applying the electrical signal along an area in the second portion of the bath to solidify the aqueous build material at one or more second locations of the bath.

19. The method of claim 15, wherein the aqueous silk fibroin is present in the bath in an amount of 5-15 weight percent, based on a total weight of the bath.

20. The method of claim 15, wherein:
the bath further comprises a leaching agent; and
the leaching agent comprises one or more of TEMED, PEG, and PEGDA.
